(19) United States
(12) Patent Application Publication
(10) Pub. No.: US 2008/0167660 A1
(43) Pub. Date: Jul. 10, 2008

(54) Suture anchor and inserter arrangement

(76) Inventors: Nathan Ryan Moreau, Portland, OR (US); Margaret Mary Sinnott, Logan, UT (US); M. Todd Miller, San Jose, CA (US)

Correspondence Address: FLYNN, THIEL, BOUTELL & TANIS, P.C., 2026 RAMBLING ROAD, KALAMAZOO, MI, 49008-1631, US

(21) Appl. No.: 11/649,726

(22) Filed: Jan. 4, 2007

(51) Int. Cl. A61B 17/04

(52) U.S. Cl. 606/104; 606/232

(57) ABSTRACT

A suture anchor and inserter arrangement, including a suture anchor configured for implanting in hard tissue, such as bone, and an inserter device for installing the anchor in hard tissue. The suture anchor includes at least one eyelet formed from suture material at a proximal end thereof, which eyelet cooperates with a working suture associated with the inserter device. The working suture is engaged with the eyelet of the implanted anchor, and can be utilized to anchor soft tissue to hard tissue.

### FIELD OF THE INVENTION

This invention relates to a suture anchor and inserter arrangement for use in fixing surgical suture to bone.

### BACKGROUND OF THE INVENTION

During some injuries, soft tissue, such as tendons or ligaments, can tear away from hard tissue, such as bone. Accordingly, it becomes necessary to reattach the soft tissue to the bone in order to facilitate the healing process. Various types of devices are used to reattach tissue, such as screws, staples and suture anchors. The instant invention relates to this latter type of attachment device.

Suture anchors may be inserted into a preformed hole made in the hard tissue, while other anchors are self-tapping. The anchors typically include an eyelet through which lengths of repair suture or working suture are threaded, which working suture is inserted simultaneously with the anchor into the hard tissue. In this regard, an inserter device or driver may be utilized in conjunction with the anchor to install or drive same into hard tissue and may carry such working sutures thereon. For the purpose of providing pull-out resistance, some anchors are exteriorly threaded, while others are ribbed or barbed to provide appropriate pull-out resistance.

U.S. Pat. No. 6,641,597 discloses a suture anchor having formed thereon an eyelet on the proximal end thereof which cooperates with repair or working suture, as mentioned above. The suture anchor according to this patent utilizes suture material to form the eyelet, and the suture material is fixed to the anchor body by insert molding. This suture anchor defines exterior ribs to maintain the anchor within the hard tissue.

U.S. Pat. No. 5,584,836 discloses a suture anchor which is exteriorly threaded to resist pull-out, and an eyelet on the proximal end of the anchor. The eyelet is rigid and is an integral component of the anchor body. U.S. Pat. No. 5,370,662 also discloses a suture anchor with an eyelet or multiple eyelets at the proximal end of the anchor which are rigid and are an integral part of the anchor body.

The suture anchor according to the invention includes an anchor body having a distal end configured for insertion into hard tissue and a proximal end spaced from the distal end. The anchor body incorporates a plurality of eyelets formed from suture material, wherein the eyelets are oriented at the proximal end for cooperation with working sutures for anchoring soft tissue to hard tissue.

Further, the suture anchor in one embodiment is formed with a pair of longitudinally-extending bores, through which the suture material is passed in order to secure same to the anchor body. This dual-bore construction is in contrast to some conventional anchors which are formed with a centrally-located bore, which dual-bore construction is believed to provide the anchor with added strength. This construction also permits the anchor to be made with a relatively small diameter, such as the range of approximately 1.5 mm to approximately 3.5 mm, for example, and additionally allows the anchor to be provided with more than one suture eyelet. Providing conventional rigid eyelets which are formed integrally with the anchor body as discussed above is difficult if not impossible on such a small scale. In addition, providing multiple eyelets allows a more even force distribution on the anchor, which can in turn reduce the possibility of structural failure and can also provide the surgeon with more options during installation.

The suture anchor according to the invention in one embodiment is configured for cooperation with an inserter device. The inserter device includes a handle for manipulating the device and an inserter shaft which supports the suture anchor at the distal end thereof. The inserter device carries working sutures, which working sutures are threaded through the eyelet or eyelets of the suture anchor. The distal end of the inserter shaft is configured to cooperatively engage and support the suture anchor.

One possible use of the arrangement according to the invention is in arthroscopic shoulder surgery, wherein the dislocation of soft tissue relative to the bone is a fairly common injury. However, this arrangement may also be utilized for the repair of small joints, such as the elbow, wrist, ankle, hand or foot. The arrangement may additionally be used to reattach small ligaments in the knee, and may even be used in bladder-neck suspension surgery.

Other objects and purposes of the invention will be apparent to persons familiar with arrangements of this general type upon reading the following specification and inspecting the accompanying drawings.

### BRIEF DESCRIPTION OF THE DRAWINGS

FIG. 1 is a fragmentary perspective view of the suture anchor and inserter arrangement according to the invention.

FIG. 2 is a fragmentary, partially-exploded perspective view of the arrangement of FIG. 1.

FIG. 3 is an enlarged, fragmentary perspective view of the proximal end of the inserter device.

FIG. 4 is an enlarged perspective end view of the proximal end of the inserter device.

FIG. 5 is an enlarged, fragmentary perspective view of the distal end of the inserter device.

FIG. 6 is a cross-sectional view of the inserter device taken generally along line 6-6 in FIG. 5.

FIG. 7 is an enlarged perspective side view of the suture anchor.

FIG. 8 is an enlarged plan view of the suture anchor.

FIG. 9 is an enlarged perspective side view of the suture anchor rotated approximately 180° from the position shown in FIG. 7.

FIG. 10 is an enlarged distal end view of the suture anchor as seen in generally in the direction of arrow B in FIG. 7.

FIG. 11 is an enlarged proximal end view of the suture anchor as seen generally in the direction of arrow C in FIG. 9.

FIG. 12 is an enlarged cross-sectional view of the suture anchor taken generally alone line 12-12 in FIG. 7.

FIG. 13 is an enlarged perspective side view of the suture anchor with the eyelets provided thereon.

FIG. 14 is a fragmentary view illustrating the suture anchor being installed within a bone using the inserter device.

FIG. 15 is a fragmentary view illustrating the inserter device being pulled away from the suture anchor.

FIG. 16 is a fragmentary view illustrating the attachment of the working sutures to soft tissue.

FIG. 17 is a fragmentary view illustrating the soft tissue fully attached to the bone.

Certain terminology will be used in the following description for convenience in reference only, and will not be limiting. For example, the words “upwardly”, “downwardly”, “rightwardly” and “leftwardly” will refer to directions in the drawings to which reference is made. The words “inwardly” and “outwardly” will refer to directions toward and away from, respectively, the geometric center and designated parts of thereof. The word “distally” will refer to the direction towards the end of the arrangement located closest to the patient, and the word “proximally” will refer to the direction towards the end of the arrangement located remote from the patient. Said terminology will include the words specifically mentioned, derivatives thereof, and words of similar import.

### DETAILED DESCRIPTION

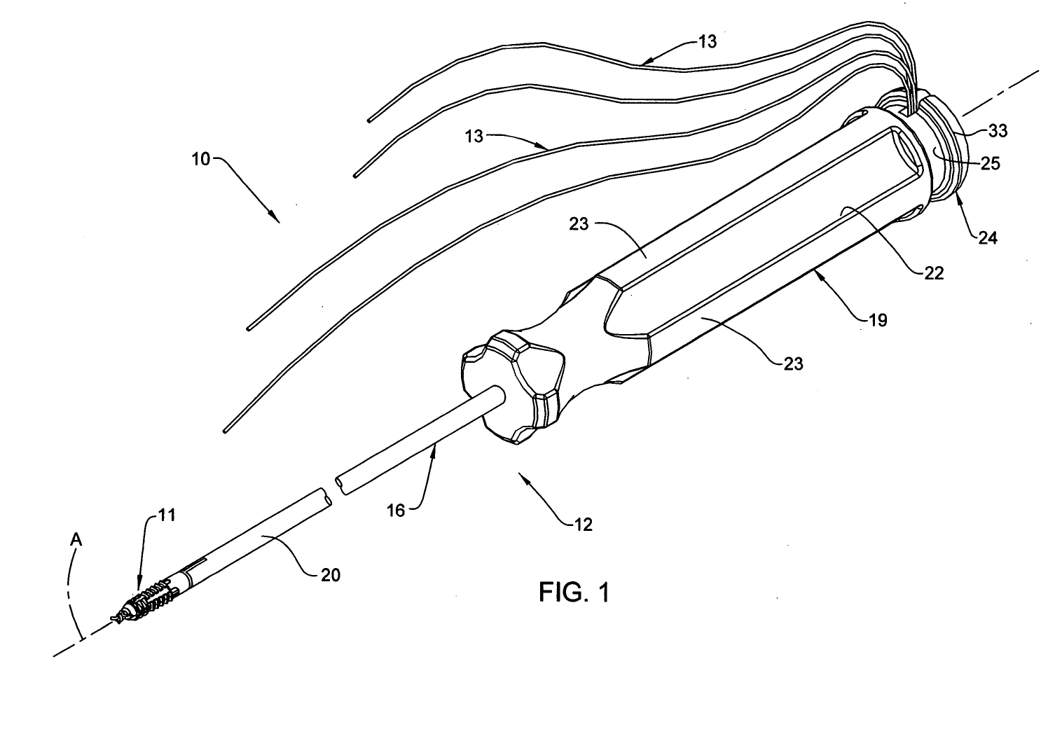

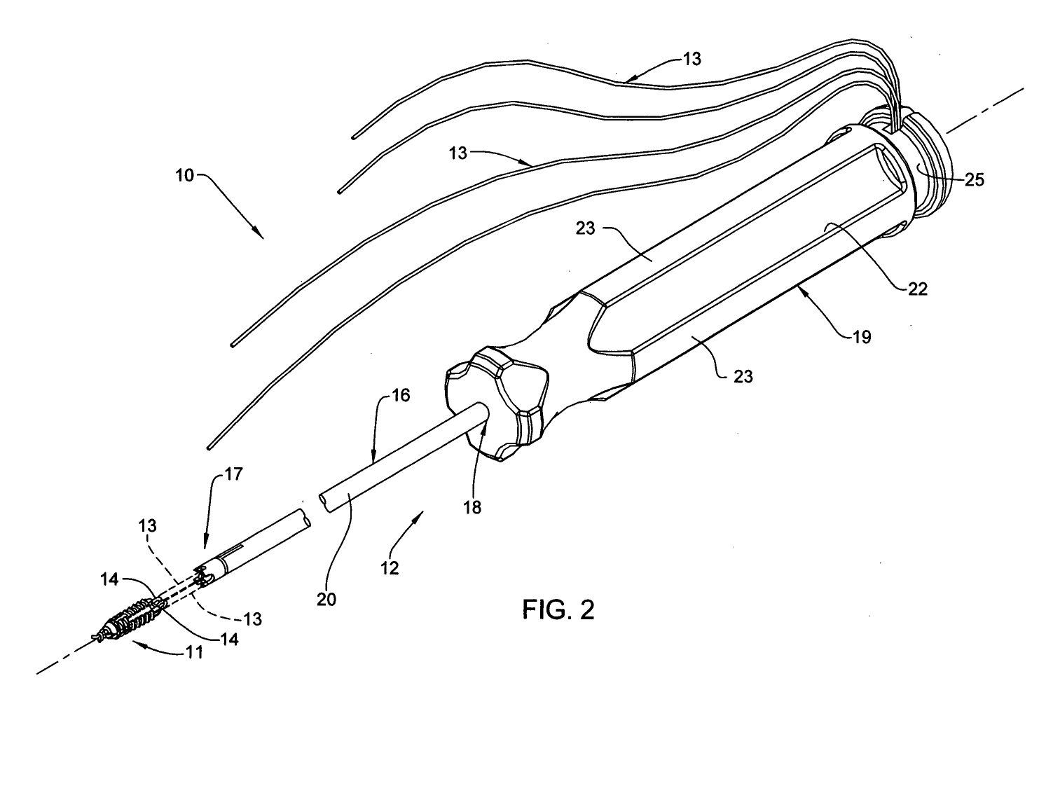

Referring to FIGS. 1 and 2, a suture anchor and inserter arrangement 10 is illustrated which is generally elongated in shape and defines a central longitudinal axis “A”. The arrangement 10 generally includes a suture anchor 11 initially supported on an inserter device 12. Working sutures 13 extend through the inserter device 12 and cooperate with eyelets 14 defined at the proximal end of the suture anchor 11.

The inserter device 12 is defined by an elongate and rigid inserter shaft 16 having a distal end 17 which engages the suture anchor 11, and a proximal end 18 fixed to a handle 19. Inserter shaft 16 includes a tubular sidewall 20 which defines a bore 21 extending throughout the longitudinal length of shaft 16 (FIG. 6). Handle 19 has an outer surface defining therein a series of axially or longitudinally extending recesses or depressions 22, wherein each circumferentially adjacent pair of recesses 22 are separated by a longitudinally extending projection 23. The alternating recesses 22 and projections 23 provide the handle 19 with a suitable gripping surface similar to a screwdriver for use when manipulating the arrangement 10 with the hand.

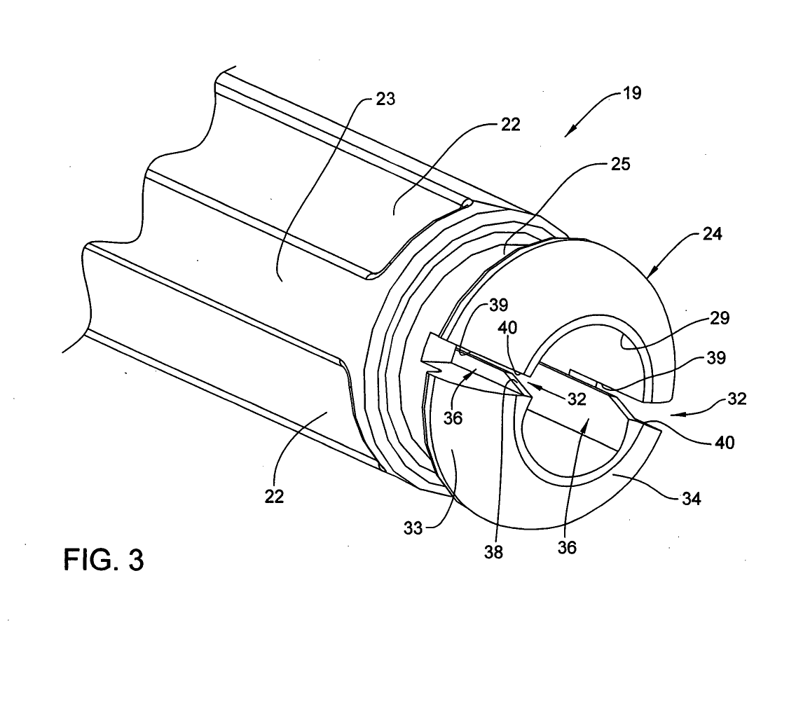

As best shown in FIGS. 3 and 4, handle 19 includes a generally annular flange 24 which defines the most proximal terminal end of handle 19. An annular recess 25 is located axially adjacent and distally of flange 24. Handle 19 defines therein a bore including a proximal bore portion 29 opening proximally or axially through flange 24, and a distal bore portion 30. Distal bore portion 30 is in communication with, and has a lesser diameter than, proximal bore portion 29 and extends distally therefrom throughout the longitudinal extent of handle 19 for communication with bore 21 of inserter shaft 20. Handle 19 additionally includes a transversely oriented wall 31 which defines the terminal distal extent of proximal bore portion 29, and through which wall 31 distal bore portion 30 opens for communication with proximal bore portion 29.

In the illustrated embodiment, a pair of slots 32 which cooperate with working sutures 13 are provided within the proximal end of handle 19 diametrically opposite one another. Slots 32 as shown are identical to one another, and only one of same will accordingly be described herein. Slot 32 opens sidewardly outwardly through an outermost side surface 33 of flange 24, axially through a terminal proximal end surface 34 of flange 24, and sidewardly inwardly for communication with proximal bore portion 29. Further, slot 32 extends distally a short distance from flange 24 so as to communicate with and open into annular recess 25 of handle 19. A pair of identical and generally planar slot-restricting walls or cleats 36 are formed on handle 23 within proximal bore portion 29 sidewardly inwardly from outermost side surface 33 of flange 24 within each respective slot 32. Wall 36 defines a first elongate and generally planar surface 37 and a second guide surface 38 which is joined to first surface 37 and angles or slopes downwardly (FIGS. 3 and 4) as surface 38 projects proximally away from surface 37. Surfaces 37 and 38 effectively provide slot 32 with a narrowed distal and generally linear section 39 and a widened proximal section 40 which opens proximally at end surface 34 of flange 24. It will be appreciated that only one wall or cleat 36 may be provided within the proximal end of handle 19, such that the slot 32 provided with wall 36 is restricted as described above and the opposite slot 32 is fully open.

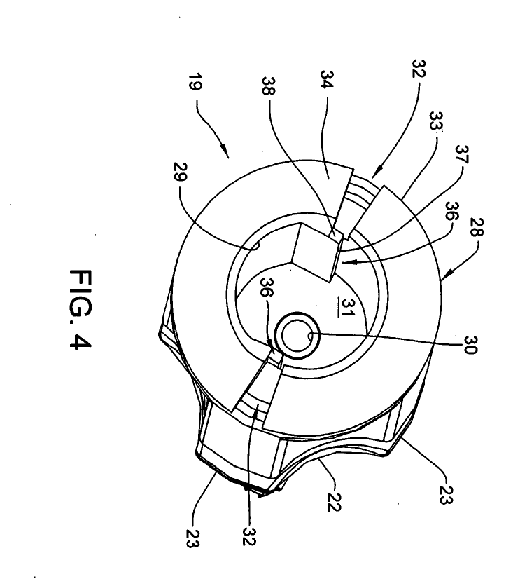

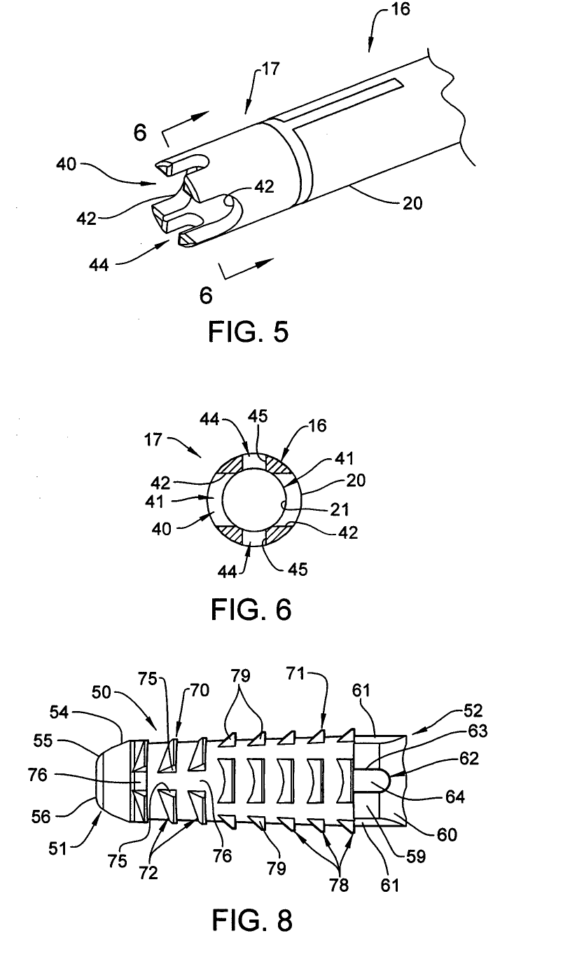

With reference to FIGS. 5 and 6, the distal end 17 of inserter shaft 16 defines therein a main slot 40 which extends transversely across the entire diameter of shaft 16 so as to define a pair of proximally extending slots 41 in each of the opposite portions of tubular sidewall 20 of shaft 16. The slots 41 are diametrically opposite one another and are each defined by an arcuately-shaped edge 42 of sidewall 20, which edges 42 are mirror images of one another. The distal end 17 of shaft 16 is also provided with a pair of anti-rotation slots 44 located diametrically opposite one another and offset circumferentially approximately 45° from slots 41. The slots 44 are each defined by an arcuately-shaped edge 45 of sidewall 20, which edges are mirror images of one another. Further, in the illustrated embodiment, slots 44 each have a circumferential width which is less than a circumferential width of the respective slots 41. Slots 41 and 44 all communicate with bore 21 of shaft 16.

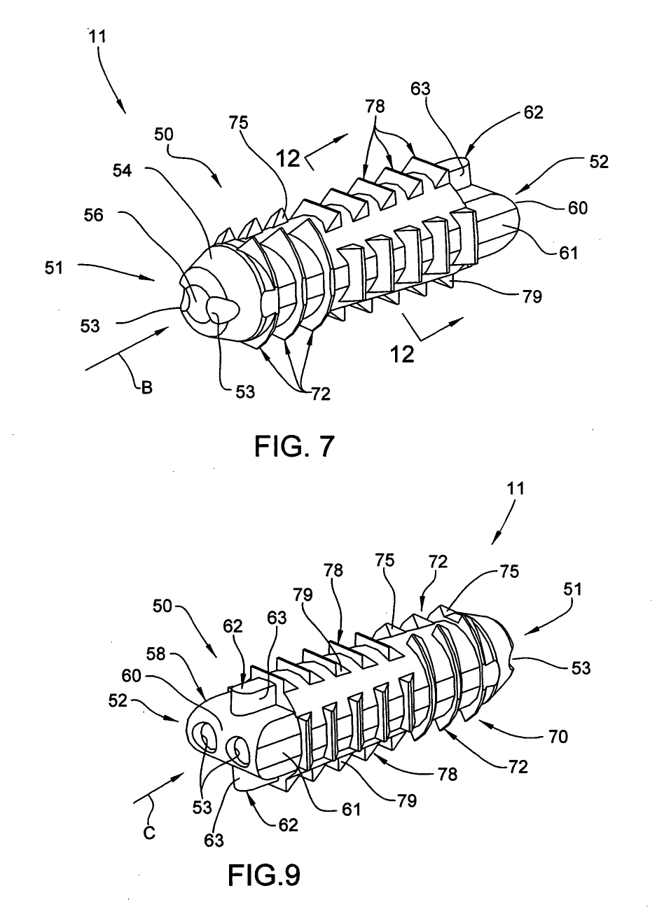

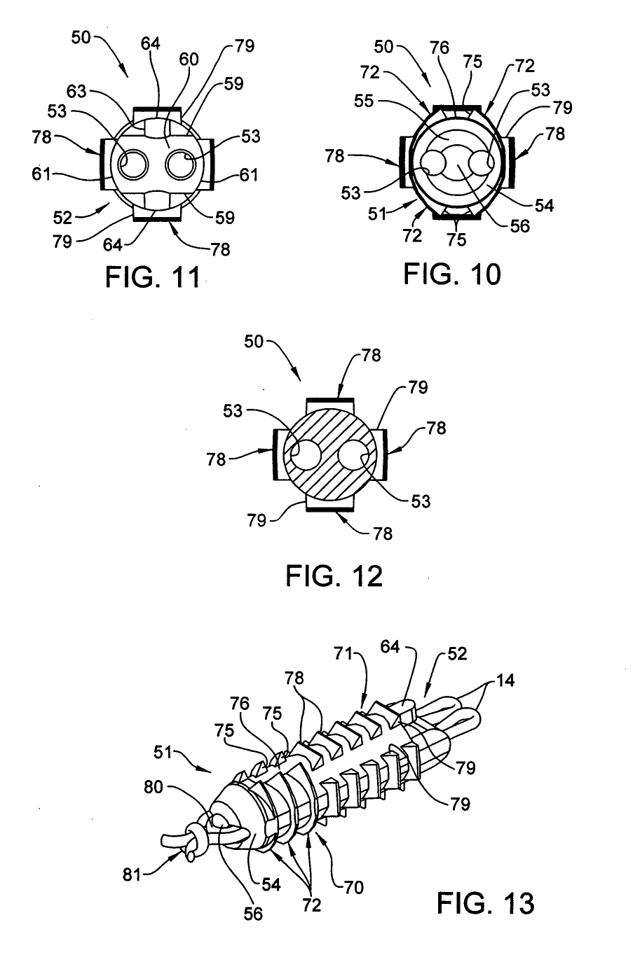

Turning now to suture anchor 11 as shown in FIGS. 7-13, same includes an generally elongate anchor body 50 including a distal end 51 which is the end first inserted into the bone and a proximal end 52 associated with the distal end 17 of inserter shaft 16. A pair of longitudinally extending bores or passages 53 extend through the entire longitudinal extent of anchor 11, and open through both the proximal and distal ends 52 and 51. Passages 53 are offset from, or located on opposite sides of, central axis A of the arrangement 10. The distal end 51 of anchor body 50 is defined by a generally truncated cone-shaped portion 54 which tapers towards central axis A as same projects distally (FIG. 8). Cone-shaped portion 54 is joined to an annular and rounded edge portion 55, which edge portion 55 is joined to a generally flat end face 56 which defines the distal most terminal end of anchor 11. The distal ends of the respective bores 53 open distally through cone-shaped portion 54, edge portion 55 and end face 56, on opposite sides of end face 56 (FIG. 10).

The proximal or head end 52 of anchor body 50 is keyed in configuration so as to cooperatively engage with the distal end 17 of inserter shaft 16. More specifically, proximal end 52 is defined by a projection or lug 58 having generally flat and generally parallel upper and lower surfaces 59, which surfaces 59 are joined to one another by a rounded end face 60 defining the proximal-most terminal end of anchor 11. The proximal ends of the respective bores 53 open proximally through rounded end face 60 (FIG. 11). Upper and lower surfaces 59 and rounded end face 60 are joined to one another by a pair of rounded side surfaces 61. A pair of identical keys or projections 62 are cantilevered outwardly from the respective upper and lower surfaces 59 of projection 58. Each key 62 defines a rounded side surface 63 which is shaped so as to engage within a respective slot 44 of inserter shaft 16, and rounded top and bottom surfaces 64. Likewise, projection 58 is sized so as to be received within main slot 40. As shown in FIG. 11, the rounded side surfaces 61 of projection 58 lie on the same circle as rounded top and bottom surfaces 64 of keys 62, and this circle is of the same or similar diameter as the outer diameter of inserter shaft 16, such that a smooth transition area is provided between the suture anchor 11 and inserter shaft 16 when the suture anchor 11 is installed thereon.

Between the proximal and distal ends 52 and 51 of anchor body 50, anchor 11 is provided with two groupings of projections, ribs or barbs which are generally wedge-shaped and have their narrow ends oriented or pointing distally. A first group of barbs 70 are located generally adjacent the distal end 51 of the anchor body 50, and a second group of barbs 71 are located generally adjacent the proximal end 52 of anchor body 50.

In the illustrated embodiment, the distal end barbs 70 are arranged in four columns, which columns extend longitudinally along the anchor body 50 and are spaced circumferentially from one another about anchor body 50. The individual barbs 72 of the distal barb group 70 each extend circumferentially along and project outwardly from the exterior surface of anchor body 50. The ends of the respective barbs 72 of the two circumferentially adjacent columns of barbs located at the upper side of the anchor body 50 and the ends of the respective barbs 72 of the two circumferentially adjacent columns of barbs located at the lower side of the anchor body 50 (FIG. 10) respectively define generally triangular end faces 75, and these end faces 75 of circumferentially adjacent pairs of barbs 72 located in circumferentially adjacent columns of barbs are disposed in facing relation with one another and angle inwardly towards one another as same project towards central axis A. A non-barbed space 76 is disposed between these faces 75. The opposite ends of these barbs 72, as barbs 72 extend circumferentially towards the respective sides of anchor body 50 and away from end faces 75, gradually diverge or project inwardly towards the outer surface of anchor body 50 and join to one another at the sides of body 50. In the illustrated embodiment, three barbs 72 are provided in each of the four columns. It should be understood, however, that a greater or lesser number of barbs 72 and/or barb columns can be provided in accordance with the invention.

The proximal end barbs 71 are also arranged in four columns, which columns extend longitudinally along the anchor body 50 and are spaced circumferentially from one another about anchor body 50 at approximately equal distances from one another. The individual barbs 78 of the proximal barb group 71 each extend circumferentially along and project outwardly from the exterior surface of anchor body 50. The ends of the respective barbs 78 of circumferentially adjacent columns of barbs define generally triangular end faces 79, and these end faces 79 of circumferentially adjacent pairs of barbs 78 located in circumferentially adjacent columns of barbs are disposed in facing relation with one another and are disposed at approximately right angles relative to one another. In the illustrated embodiment, five barbs 78 are provided in each column, although a greater or lesser number of barbs and/or barb columns may be utilized in accordance with the invention.

In the illustrated embodiment, the respective columns of distal end barbs 72 are offset by approximately 45° relative to the respective columns of proximal end barbs 78, which allows for staggered contact of the points or peaks of the barbs 72, 78 with the bone or hard tissue surface.

FIG. 13 illustrates the suture anchor 11 with the eyelets 14 provided thereon. In this regard, the eyelets 14 are formed from a single strand or length of suture material which is inserted through the longitudinal bores 53 defined in the anchor body 50 to secure the suture material thereto. In accordance with one method of assembling suture material to anchor 11, and with reference to FIGS. 7, 9 and 13, one free end of the suture strand is inserted into and through one of the bores 53 at the distal end 51 of anchor 11 and is threaded through this bore 53 until same emerges at the opposite end of bore 53 at the proximal anchor end 52. The strand is then looped over itself to form one eyelet 14 at proximal end 52 and projecting outwardly from proximal end 52, and the strand is then threaded back through the same bore 53, extended transversely across end face 56 of distal end 51 to form distal strand section 80, and then inserted into the opposite bore 53 back to the proximal end 52 and exits bore 53 thereat. At this location, another eyelet 14 is formed by looping the strand over itself and the strand is then inserted back through the same bore 53 until same emerges at the distal end 51 of anchor body 50. The two free ends of suture are then tied at the distal end to form a knot 81 adjacent and in generally overlapping relation with distal strand section 80. Further, an alternative method of assembling the suture to anchor 11 would be to insert two suture loops through the anchor 11 starting at the distal end 51 thereof through the respective bores 53, and then tying the free ends into knot 81 at distal end 51.

One method of assembling the suture anchor 11 onto the inserter device 12 is as follows. Free ends of two separate working or repair sutures 13 are inserted into the proximal end of inserter device 12 through proximal bore portion 29, into distal bore portion 30, and through inserter shaft bore 21. These free ends of the working sutures 13 are inserted or threaded through the respective eyelets 14 disposed at the proximal end of anchor body 50 (shown in dotted lines in FIG. 2), and then looped back through the inserter device 12 until same emerge at proximal bore portion 29 thereof. Alternatively, instead of utilizing a pair of working sutures 13, a single working suture could be threaded through both eyelets 14. The keys 62 at the proximal end of anchor 11 are then circumferentially or rotationally aligned with the respective slots 44 at the distal end of inserter shaft 16, and the proximal end of the anchor 11 is inserted into the open end of the inserter shaft 16 so that keys 62 are engaged within the respective slots 44, and so that the opposite sides 61 of projection 58 are engaged within the respective slots 41. The free ends of the working sutures 13 located adjacent handle 19 are then pulled in a proximal direction so as to tension the working sutures 13, and the working sutures 13 may then be pulled transversely or sidewardly relative to handle 19 to engage the working sutures 13 within the narrow portion 39 of one of the slots 32 so as to maintain the sutures 13 in a fixed position relative to inserter device 12. If desirable or necessary, for example for storage purposes, the free ends of working sutures 13 may be wrapped or coiled around handle 19 and stored within annular recess 25, and the free ends fixed in place within the opposite slot 32. Alternatively, if only one of the slots 32 defined in handle 19 is provided with a restricting wall 36 as described above, then the working sutures 13 may exit the proximal end of handle 19 and be pulled sidewardly or transversely relative thereto so as to engage the working sutures 13 within the open or non-restricted slot 32. The free ends of sutures 13 may then be wrapped or coiled around handle 19 within recess 25 and then locked within the opposite restricted slot 32. This embodiment of handle 19 may be easier to manipulate, since the open or non-restricted slot 32 allows the sutures 13 exiting handle 19 to track within such slot 32 while same are coiled about handle 19.

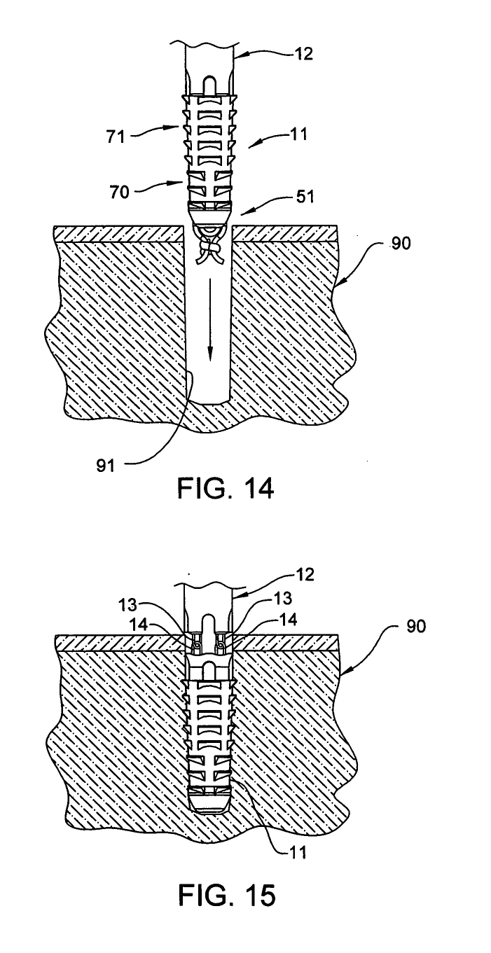

The suture anchor 11 is intended for implanting within hard tissue, such as bone 90. One method of implanting anchor 11 will be described with reference to FIGS. 14-17. First, a hole 91 is drilled into the cortical and cancellous layers of the bone 90, which hole 91 is slightly undersized relative to the outer diameter of the suture anchor 11. With the suture anchor 11 installed on the inserter device 12 as discussed above, the distal end 51 of the anchor 11 is aligned with the hole 91 and the anchor 11 is pressed or tapped into hole 91 utilizing device 12 (FIG. 14). Once the anchor 11 is located at the desired depth within hole 91, the inserter device 12 is pulled in a proximal direction away from the anchor 11 (FIG. 15) to unseat the anchor 11. In this regard, the working sutures 13 would be released from narrow slot portion 39 of handle 19 prior to the aforementioned step, so as to allow working sutures 13 to move freely relative to the inserter device 12 as the anchor 11 is deployed therefrom. Continued movement of the inserter device 12 in a proximal direction frees the working sutures 13 from the device 12, so that the surgeon can use the sutures 13 to anchor soft tissue 92 to the bone 90.

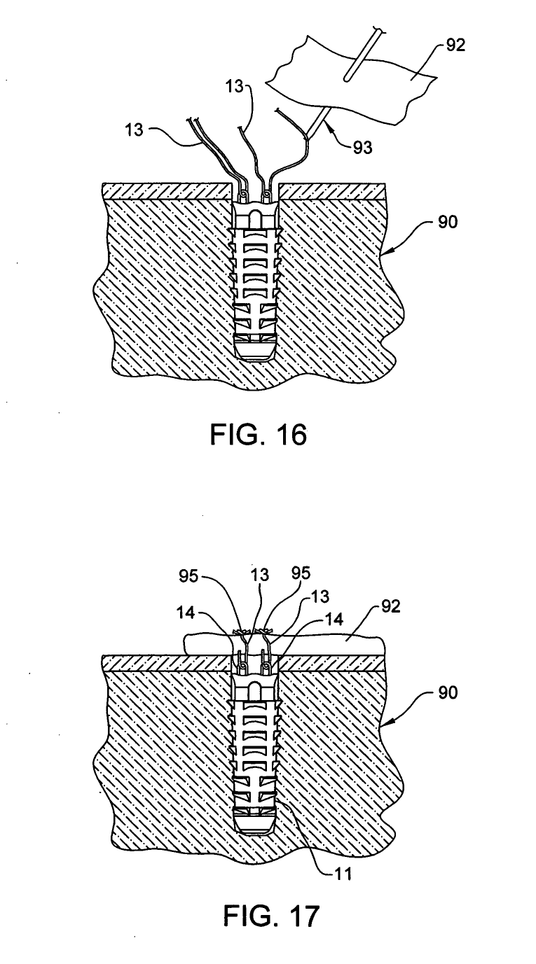

As shown in FIG. 16, the surgeon utilizes a suitable surgical tool 93 to grasp one of the working sutures 13 and pull same through the soft tissue 92. The same step is performed with the opposite working suture 13. The two free ends of each of the working sutures 13 now extend around and through a portion of the soft tissue 92, and the surgeon forms sliding knots 95 in the working sutures 13 which knots are moved down the working suture 13 to cinch the soft tissue 92 against the bone 90 at the location of anchor 11 (FIG. 17). The excess length of each working suture 13 is then removed, if necessary. Once the suture anchor 11 is seated within the bone 90 within hole 91, the barbs 70, 71 will prevent proximal movement of the anchor 11 out of hole 91 so that the anchor 11 will remain firmly fixed within the bone 90. It will be appreciated that the 45° offset construction of the distal and proximal columns of barbs provides improved pull-out resistance.

The suture anchor 11 according to the invention is not formed with a centrally located bore as are many conventional anchors, and instead is formed with two longitudinal bores or passages 53 as described above. This structure is believed to result in a mechanically strong anchor, and allows the anchor to be made small in diameter. In accordance with one embodiment, the suture anchor 11 according to the invention may have an outer diameter in the range of approximately 1.5 mm to approximately 3.5 mm for use in situations which require a relatively small anchor, for example in shoulder surgery, small joint surgery, or repair of small knee ligaments. However, it will be appreciated that the arrangement according to the invention may be utilized in other types of surgeries which may require a larger-diameter anchor, such as in the range of approximately 3.5 mm to approximately 10 mm, for example. When suture anchor 11 according to the invention is to be utilized in arthroscopic shoulder surgery, wherein the dislocation of soft tissue relative to the bone is a fairly common injury, an undersized hole, for example of approximately 2 mm-2.5 mm diameter, can be formed in the bone as described above for use with an approximately 3.5 mm diameter suture anchor.

Further, the two-bore construction of the anchor 11 allows the anchor 11 to be provided with more than one suture eyelet, and the formation of the eyelets 14 from a length of suture make it possible to place two flexible eyelets on such a small anchor. Providing conventional rigid eyelets is not believed feasible on such a small scale. Still further, separating the load of fastening soft tissue between two eyelets instead of relying on only one eyelet permits more even load distribution, which can reduce the risk of structural failure of an eyelet. In addition, providing more than one eyelet makes it easier to cinch down the working sutures to the bone, and gives the surgeon more flexibility during surgery.

It is also within the scope of the instant invention to provide multiple eyelets 14 on the anchor 11, which anchor 11 is provided with exterior threads in place of the barbs described herein. In accordance with a further embodiment, the anchor described herein can be provided with a single bore or passage in place of bores 53. This single bore may be centrally located along axis A, or offset sidewardly from axis A. In this embodiment, the suture material can be tied with a knot so as to form two or more eyelets which emerge from the body of the knot. The eyelets can then be grasped and pulled through the single bore defined in the anchor in a direction distal to proximal until the eyelets emerge from the proximal or head end of the anchor. The suture material would be fixed to the anchor body by sizing the bore and knot such that the knot is too large to pull completely through the proximal end of the anchor body, and thus is wedged within the bore to support the load placed on the eyelets and to fix the suture material to the anchor body.

The suture anchor 11 according to the invention may be constructed of any suitable rigid material, such as plastic or metal, and also may be constructed of bio-absorbable material or non-absorbable material. In this regard, one example of nonabsorbable plastic which may be utilized is PEEK, and one example of nonabsorbable metal which may be utilized is titanium. One example of an absorbable plastic which may be utilized is PLLA. Composite materials may also be used for both bio-absorbable and non-absorbable applications, such as PLLA/HA, which is a type of ceramic. It will be appreciated that other types of materials may be utilized in accordance with the invention, and the above are presented only as examples.

Although a particular preferred embodiment has been disclosed in detail for illustrative purposes, it will be recognized that variations or modifications of the disclosed apparatus, including the rearrangement of parts, lie within the scope of the present invention.

What is claimed is:

1. A suture anchor for implanting into hard tissue, such as bone, said anchor comprising an anchor body having a distal end configured for insertion into hard tissue and a proximal end spaced from said distal end, said anchor body including a plurality of eyelets formed from suture material, said eyelets being disposed at said proximal end for cooperation with working suture for anchoring soft tissue to hard tissue.

2. The suture anchor of claim 1, wherein said anchor body comprises a plurality of barbs which project outwardly therefrom for securing said anchor within bone.

3. The suture anchor of claim 1, wherein said anchor body defines a central longitudinal axis, and a bore is disposed in said anchor body offset from the axis through which said suture material forming said eyelets extends.

4. The suture anchor of claim 1, wherein said anchor body defines a central longitudinal axis, and a pair of bores are disposed offset from the axis and open through said proximal end of said anchor body, wherein respective portions of said suture material are disposed within said bores and two of said plurality of eyelets project outwardly from said bores.

5. The suture anchor of claim 1, wherein said suture material forming said eyelets is fixedly secured to said anchor body by means other than insert molding.

6. The suture anchor of claim 1, wherein said proximal end of said anchor body is configured for cooperative engagement with a distal end of an inserter device for driving said anchor into hard tissue.

7. The suture anchor of claim 6, wherein said anchor defines a central longitudinal axis, and said proximal end of said body defines a projection which extends proximally and is oriented transversely relative to the axis, and a key cantilevered from said projection and oriented transversely relative thereto.

8. The suture anchor of claim 7, wherein said projection has portions which are disposed inwardly from an outer diameter of said anchor body.

9. A suture anchor for securing tissue to bone, said suture anchor comprising a body having distal end, a proximal end spaced from said distal end, a non-threaded exterior surface including a plurality of projections cantilevered from said outer surface for engaging within bone and fixing said suture anchor thereto, an eyelet formed from suture material and disposed at said proximal end, said suture material being secured to said anchor body to prevent movement relative thereto but non-fixedly engaged with an interior of said anchor body.

10. The suture anchor of claim 9, wherein said body defines a longitudinally extending bore therein in which said suture material is disposed, said suture material being secured to said anchor body by passing slidingly through said bore.

11. The suture anchor of claim 9, wherein a pair of longitudinally extending bores are defined within said body and open through said proximal end thereof for cooperation with working sutures of an inserter device, and a pair of said eyelets formed from suture material are disposed at said proximal end, said suture material having portions extending through the respective bores and exiting said bores at said proximal end to form said eyelets.

12. The suture anchor of claim 11, wherein said bores additionally open through said distal end of said body, and said suture material is a single strand of suture material having a pair of free ends which exit said body at said distal end and are secured together thereat.

13. The suture anchor of claim 9, wherein said projections are barb-shaped and are disposed along said body between said proximal and distal ends.

14. The suture anchor of claim 9, wherein said body has an outer diameter of approximately 3.5 mm.

15. A suture anchor for securing soft tissue to hard tissue, said suture anchor comprising a generally elongate body defining a central longitudinal axis, a distal end configured for insertion into hard tissue, and a proximal end spaced from said distal end and configured for cooperation with an inserter device for installing said suture anchor into hard tissue, a pair of bores extending longitudinally through said body and opening adjacent said proximal end, and suture material disposed within the respective bores and emerging through said bores at said proximal end of said body to form a pair of eyelets which cooperate with working suture associated with an inserter device.

16. The suture anchor of claim 15, wherein said bores additionally open adjacent said distal end of said body, and said suture material has a pair of free ends which emerge through said bores at said distal end and are secured together at said distal end.

17. The suture anchor of claim 15, wherein a plurality of generally wedge-shaped barbs are disposed on said body between said proximal and distal ends for securing said suture anchor within hard tissue.

18. The suture anchor of claim 17, wherein first ones of said barbs are disposed along an outer diameter of said body in circumferentially-spaced relation with one another, and second ones of said barbs are disposed along an outer diameter of said body in circumferentially-spaced relation with one another and in axially-spaced relation with said first barbs, said second barbs being circumferentially offset from said first barbs.

19. A suture anchor for implanting in hard tissue, such as bone, said anchor including a body having a distal end and a proximal end spaced therefrom, suture material secured to said body and being slidingly inserted thereinto to form an eyelet at said proximal end of said body for cooperation with working suture, said body having an exterior surface defining therein non-threaded and spaced-apart projections configured for fixing said suture anchor within hard tissue.

20. The suture anchor of claim 19, wherein said body defines a bore therein and said suture material extends through said bore and emerges at said proximal end to form said eyelet.

21. The suture anchor of claim 20, wherein said body defines therein a pair of longitudinally-extending bores which open at both said proximal and distal ends of said body, said suture material projecting outwardly through said bores at said proximal end to form a pair of said eyelets, said suture material having a pair of free ends projecting outwardly through the respective bores at said distal end and being secured to one another thereof.

22. The suture anchor of claim 21, wherein said suture material is a single strand of suture material.

23. A combination suture anchor and inserter device for anchoring soft tissue to hard tissue, said combination comprising:
an inserter device including a handle for manipulating said inserter device and an elongate shaft fixed to and extending distally from said handle, said shaft having a distal end remote from said handle; and
a suture anchor having a distal end configured for insertion into hard tissue and a proximal end spaced therefrom, and a pair of eyelets formed from suture material disposed at said proximal end, said proximal end of said anchor being configured for cooperative engagement with said distal end of said inserter device.

24. The combination of claim 23, wherein one of said proximal end of said anchor and said distal end of said shaft is engaged within the other of said proximal end of said anchor and said distal end of said shaft.

25. The combination of claim 23, wherein one of said proximal end of said anchor and said distal end of said shaft is keyed relative to the other of said proximal end of said anchor and said distal end of said shaft so as to prevent relative rotation between said anchor and said shaft.

26. The combination of claim 23, wherein said inserter device defines a central longitudinal axis and said shaft is generally tubular in configuration, said proximal end of said anchor defines thereon a projection and said distal end of said shaft defines therein a slot extending transversely of said shaft and opening distally, said projection being sized so as to engage within said slot.

27. The combination of claim 26, wherein said anchor includes a key cantilevered from said projection and oriented transversely relative to the axis and relative to said projection, said slot is a first slot and a distal end of said shaft defines therein a second slot which is circumferentially offset from said first slot, said key being sized so as to engage within said second slot.

28. The combination of claim 23, wherein said shaft is tubular and working sutures extend from said handle and into an interior of said shaft, said working sutures exiting said distal end of said shaft and being engaged with said eyelets of said anchor.

29. The combination of claim 23, wherein said anchor has a body which defines said distal and proximal ends of said anchor, said anchor including a plurality of generally wedge-shaped barbs disposed along said body between said distal and proximal ends.
